(19) United States
(12) Patent Application Publication
(10) Pub. No.: US 2026/0365373 A1
(43) Pub. Date: Nov. 19, 2026

(54) FLUID CONDUIT HOLDER AND ASSOCIATED METHODS

(71) Applicant: Vitrolife Sweden AB, Göteborg (SE)

(72) Inventors: Csaba Pribenszky, Viby (DK); Erik Zotterman, Västra Frölunda (SE)

(21) Appl. No.: 19/133,524

(22) PCT Filed: Nov. 29, 2023
(86) PCT No.: PCT/EP2023/083532

§ 371 Date: May 28, 2025

(30) Foreign Application Priority Data
Nov. 30, 2022 (GB) 2218022.8

(51) Int. Cl. B65D 25/10; B65D 85/20

(52) U.S. Cl. CPC B65D 25/103; B65D 85/20

(57) ABSTRACT

A fluid conduit holder includes a support and a guide, wherein the support is configured to receive and locate a fluid conduit with respect to the guide in use, such that the guide provides a tapering path to a lumen of the fluid conduit.

### FIELD

The present invention relates to a fluid conduit holder. The fluid conduit holder is for housing a fluid conduit, such as a micropipette, to assist in delivering fluid e.g. operating liquid (such as that disclosed in WO2021094588) and/or sperm to the fluid conduit, which may be used e.g. for Piezo-mediated microinjection, or Piezo-mediated intracytoplasmic sperm injection (Piezo-ICSI).

The present invention further relates to methods of filling a fluid conduit using a fluid conduit holder, e.g. for fertilising an oocyte in vitro by Piezo-ICSI.

### BACKGROUND

Male factor infertility is responsible for the inability of approximately 50% of couples to conceive. Many procedures were developed in the 1980s to address fertilization failure due to dysfunctions of the male gamete including injecting a single sperm cell (spermatozoa) under the zona pellucida of the oocyte, with very limited success rates. However, it was during the performance of subzonal injection of an oocyte that the oolemma was accidentally breached and the spermatozoon was delivered into the ooplasm, subsequently establishing the development of intracytoplasmic sperm injection as it is performed still today in humans.

Intracytoplasmic sperm injection (ICSI) is an in vitro fertilization procedure in which a sperm cell is injected directly into the cytoplasm of an oocyte. ICSI is performed in a culture dish under a microscope by a human operator using micromanipulators and micropipettes to handle the oocyte and sperm. A fine glass micropipette (holding pipette) stabilizes a mature oocyte with gentle suction power applied by a microinjector whilst from the opposite side a thin, spiked glass micropipette (injection micropipette) is used to collect a single sperm, having immobilised it by hurting its tail with the point of the micropipette. Mechanical force applied by the operator through the micromanipulators is used to pierce the zona pellucida and plasma membrane (oolemma) of the oocyte with the injection micropipette. The sperm is then injected into the cytoplasm of the oocyte. ICSI has been responsible for over two million babies worldwide. However, ICSI is a labour-intensive technique, requiring a highly skilled operator, and there is still room for improvements with regards to standardisation of the technique and success rate.

An alternative oocyte micro-injection method called “Piezo-mediated” ICSI (Piezo-ICSI) was developed for mouse oocytes in 1995, as conventional ICSI was not solving the issues related to fertilisation in mouse in vitro fertilisation. Piezo-ICSI resembles conventional ICSI (c-ICSI) in that a micropipette containing a single sperm pierces an oocyte to deliver the sperm to the oocyte cytoplasm. However, where in c-ICSI the zona pellucida and oolemma are pierced by mechanical force applied by the operator through micromanipulators to the micropipette, in Piezo-ICSI a piezoelectric effect is used to pierce the oocyte. The piezoelectric effect is the phenomenon that certain materials accumulate electric charge in response to mechanical stress, and conversely mechanical stress can be induced by application of electricity. The piezoelectric effect has many technological applications. In the case of Piezo-ICSI, a brief “Piezo pulse” is applied to the micropipette containing the sperm to produce ultra-fast, sub-micron forward momentum of the micropipette. This precise and rapid movement is used to pierce the zona pellucida and oolemma in a manner that is less stressful to the oocyte than the mechanical force used in c-ICSI.

However, ICSI and Piezo-ICSI remain labour-intensive techniques, requiring a highly skilled operator, and the present invention seeks to ameliorate this.

### BRIEF SUMMARY OF THE INVENTION

The present invention provides a fluid conduit holder comprising a support and a guide, wherein the support is configured to receive and locate a fluid conduit with respect to the guide in use, such that the guide provides a tapering path to a lumen of the fluid conduit.

The present invention further provides a method of filling a fluid conduit using a fluid conduit holder, the holder comprising a support and a guide, the method comprising:
i. locating the fluid conduit with respect to the guide such that the guide provides a tapering path to a lumen of the fluid conduit; and
ii. at least partially filling the fluid conduit with fluid via the guide.

The present invention further provides additional embodiments as claimed in the dependent claims.

The claimed invention generally provides a subsystem suitable for use in a fluid delivery system, or configured for use in a fluid delivery system. The subsystem may generally form part of a fluid delivery system and in particular may form part of a fluid delivery system for in vitro fertilisation.

The claimed invention beneficially assists an operator in delivering fluid (which may comprise sperm and/or an operating fluid for specific applications) into a fluid conduit such as a micropipette (or microcapillary pipette), which may be glass and may be used e.g. for Piezo-mediated microinjection, or Piezo-mediated intracytoplasmic sperm injection (Piezo-ICSI), which can require significant control and precision. The operating liquid is typically filled in from the back (back-filled). The sperm or other cells are normally in a holding medium and the sperm holding medium is typically inserted from the front. The claimed invention may assist with filling from either or both ends of the fluid conduit. The claimed invention may thus reduce preparation time and reduce the likelihood of risks and accidents, thereby reducing wastage and improving success rates.

The fluid conduit may be shipped in the holder, reducing the risk of damage to the conduit, which may be made of glass thus be fragile, and/or enabling easy removal for attachment to a micromanipulator.

### BRIEF DESCRIPTION OF THE FIGURES

Embodiments of the disclosure will now be described, by way of example only, with reference to the accompanying drawings, in which:

FIG. 1 is a schematic perspective view of a first fluid conduit holder in accordance with some embodiments of the disclosure.

FIG. 2 is a schematic perspective view of a package for a fluid conduit holder in accordance with some embodiments of the disclosure.

FIGS. 3a and 3b are schematic, cross-section views of the interface between a fluid conduit holder and a fluid conduit in accordance with some embodiments of the disclosure.

FIG. 4 is a schematic perspective view of a second fluid conduit holder in accordance with some embodiments of the disclosure.

FIGS. 5a and 5b are photographs of the second fluid conduit holder in accordance with some embodiments of the disclosure.

FIGS. 6a and 6b are photographs of a third fluid conduit holder in accordance with some embodiments of the disclosure.

### DETAILED DESCRIPTION OF THE DISCLOSURE

Aspects and features of certain examples and embodiments are described herein. Some aspects and features of certain examples and embodiments may be implemented conventionally and these are not described in detail in the interest of brevity. It will thus be appreciated that aspects and features of apparatuses and methods discussed herein which are not described in detail may be implemented in accordance with any suitable conventional techniques.

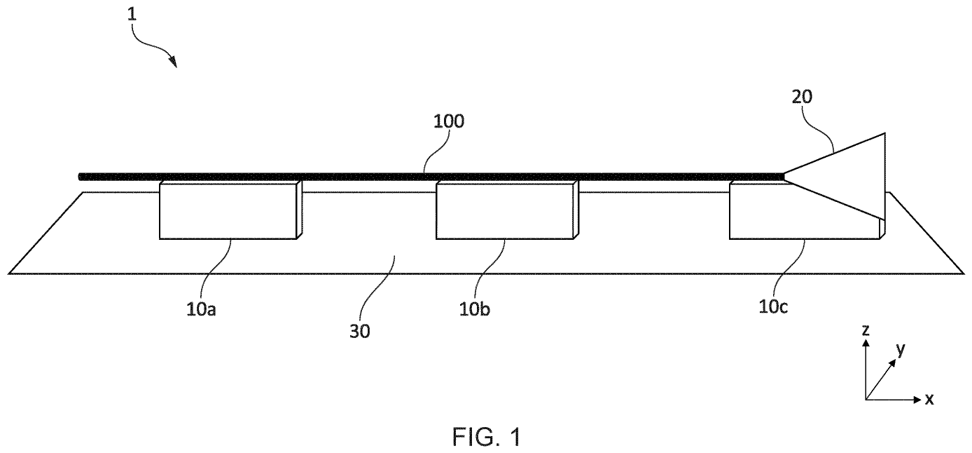

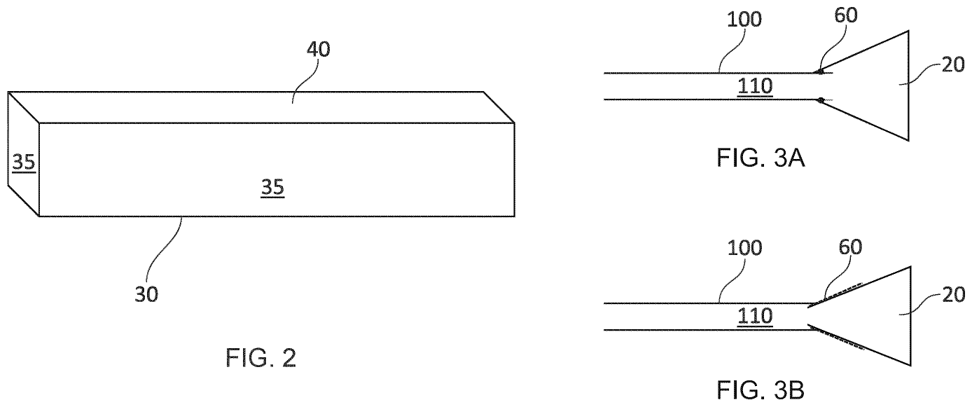

FIG. 1 is a schematic perspective view of a first fluid conduit holder 1 in accordance with some embodiments of the disclosure. FIG. 1 shows a fluid conduit holder 1 comprising three axially-aligned rectangular block supports 10a, 10b, 10c for receiving and supporting a fluid conduit 100, and a guide 20. The fluid conduit 100 may comprise a needle, a syringe or a pipette, particularly a micropipette, although the fluid conduit 100 itself is not key to the invention. The supports 10 comprise a first support 10a distal from the guide 20, plus second and third supports 10b, 10c, where the guide 20 sits on the third support 10c and the second support 10b is between the first and third supports, as an intermediate support. In the example of FIG. 1, the supports 10 each comprise an axial recess (not shown) such as a channel to receive a part of the fluid conduit 100, and are fixed to a base 30 of a protective sealing package for the holder 1, which is described later with reference to FIG. 2.

The guide in FIG. 1 comprises a funnel 20 adjoined to the third support 10c. When the holder 1 receives the fluid conduit 100 in or on the supports 10 in use, the supports 10 locate the fluid conduit 100 with respect to the guide 20 such that the guide 20 provides a tapering (narrowing) path to a lumen 110 of the fluid conduit 100. The guide 20 is configured to guide/direct a user attempting to insert a vessel containing fluid to be back-filled into the lumen 110 of the fluid conduit 100, so that the vessel can more easily be inserted into the lumen 110 and the user can fill the fluid conduit 100 with the fluid from the vessel.

The guide 20 comprises a tapering guide, providing a narrowing path to the lumen 110 of the fluid conduit 100. Preferably, the guide 20 is configured to provide a tapering path that guides towards the lumen 110 in at least two perpendicular axes. In FIG. 1, the funnel guide 20 tapers towards the lumen 110 in two perpendicular axes (i.e. the y, z axes perpendicular to the x-axis of extent of the fluid conduit 100), and thus provides a path to the lumen 110 that is guided toward the lumen 110 (or restricted away from the lumen 110) in 4 cartesian degrees of freedom (i.e. +/−y and +/−z). In other words, an object in the funnel 20, such as a vessel to be inserted into the lumen of the fluid conduit 100, is restricted in movement within the funnel 20 in 4 cartesian degrees of freedom, thus is guided to the lumen 110.

The funnel guide 20 in FIG. 1 is also configured to axially align with the fluid conduit 100 when the conduit 100 is received in the holder 1, and a lumen of the funnel guide 20 is substantially concentric with the lumen 110 of the fluid conduit 100. In embodiments where the supports 10 comprise a recess for receiving the fluid conduit 100, a lumen of the guide 20 may be substantially concentric with a lumen of the recess, to provide suitable alignment when the fluid conduit 100 is received therein.

In the example of FIG. 1, the guide comprises a funnel 20, which beneficially tapers in two perpendicular axes and guides (limits) movement in 4 cartesian directional (x, y, z) degrees of freedom (allowing free movement only in the axial direction, through the funnel 20, restricting movement away from the lumen 110 when proximal thereto). More generally, the guide 20 may comprise a funnel or cone, or a conical, frustoconical, concave, convex or tapering surface, such as a part of a funnel or cone (e.g. a half-funnel). In some embodiments, the guide 20 provides a tapering path that guides towards the lumen 110 in at least two perpendicular axes and/or provides a path to the lumen 110 that is guided toward the lumen 110 (or restricted away from the lumen 110) in at least 3 cartesian degrees of freedom.

The guide 20 may be a separate element to the supports 10, as in FIG. 1, or may be integrated into one or more of the supports 10, as shown in FIGS. 6a-b, described later. The guide 20 is sized appropriately to suit the fluid conduit 100 to be used and the present application is particularly concerned with holders suitable for micro-size fluid conduits 100 such as P10, P20 and P50 micropipettes, suitable for volumes of 0.5-10 μl, 2-20 μl and 5-50 μl respectively. For Piezo-ICSI, the amount of operating liquid that is backfilled into the micropipette is between 10 and 20 μl in most cases. In some embodiments, the fluid conduit 100 has a volume of substantially 0.5-50 μl, 2-30 μl, 5-25 μl, 10-20 μl or 15 μl and/or may have an inner diameter of substantially 0.5-20 μm, 1-10 μm, 2-8 μm, 3-7 μm, 4-6 μm, 3.5-5.5 μm or 4.5-5.5 μm. For such embodiments, the guide 20 may comprise a narrow end having an opening dimension (e.g. diameter for a funnel, width or height for a curved surface) that is substantially the same as that of the inner diameter of the fluid conduit 100 in use, or may be slightly larger (to aid insertion but without unduly restricting the inner diameter of the fluid conduit 100), or slightly smaller (potentially providing a direct path into the lumen 110 of the fluid conduit 100). In some embodiments, the narrow end opening dimension of the guide 20 may thus be substantially 0.5-50 μm, 1-25 μm, 2-20 μm, 3-15 μm, 3-10 μm, 4-6 μm, 4.5-5.5 or 3-5 μm.

During the ICSI procedure, the sperm holding medium is aspirated from the front end, typically ~2-10 μl. In piezo ICSI, first the micropipette is backfilled typically by ~15 μl of operating liquid, then it is pulled down to the front tip. After this, the sperm holding medium is aspirated from the front tip, so that it is in direct contact with the operating liquid.

The present invention thus aids an operator with filling a fluid conduit 100 by providing a fluid holder 1 which provides a tapering path to the lumen 110 of the fluid conduit 100 in use, when the fluid conduit is located in the holder 1. In use, the operator locates the fluid conduit 100 with respect to the guide 20 (e.g. substantially abutting the guide 20, or inserting the guide 20 into the conduit 100, or the conduit 100 into the guide 20) such that the guide 20 provides a tapering path to the lumen 110 of the fluid conduit 100, aiding the insertion of a vessel containing fluid to be filled into the lumen 110 of the fluid conduit 100. In some embodiments, the guide 20 may be fluidly connected to an end of the fluid conduit 100 in use.

FIG. 2 is a schematic view of a package for a fluid conduit holder 1 in accordance with some embodiments of the disclosure. The package comprises a base 30, side walls 35 and a removable protective tear-off film lid 40, covering the supports 10 and the guide 20 within. The package may provide a secure, sterile housing for the holder 1. The operator removes the film lid 40 to reveal the holder 1, ready for use.

FIGS. 3a and 3b are schematic, cross-section views of the interface between the fluid conduit holder 1 and a fluid conduit 100 in accordance with some embodiments of the disclosure. As shown in FIGS. 3a-3b, in use, in some embodiments, the fluid conduit 100 may be received in the guide 20, as in FIG. 3a; or the guide 20 may be received in the fluid conduit 100, as in FIG. 3b. The guide 20 is sized appropriately for the intended fluid conduit 100 and intended interface method. The guide 20 may be interchangeable/replaceable and multiple guides 20 of different sizes may be provided, e.g. in the package. In some embodiments, the fluid conduit 100 and/or the guide 20 is keyed on a suitable (internal/external) surface to provide a more secure engagement mechanism. In FIG. 3a, the fluid conduit 100 is received in the narrow end of the guide 20 and the guide 20 abuts or optionally secures around the outside of the fluid conduit 100, e.g. with a compressible interference fit. As shown in FIG. 3a, the guide 20 (alternatively the fluid conduit 100) may comprise a sealing element 60, such as an o-ring, configured to aid fluid sealing between the fluid conduit 100 and the guide 20. In FIG. 3b, the guide 20 is received within the lumen 110 of the fluid conduit 100. As shown in FIG. 3b, the guide 20 may comprise a sealing element 60, such as a compressible, impermeable material on the outer surface thereof, to aid fluid sealing between the fluid conduit 100 and the guide 20. Alternatively, the fluid conduit 100 may be provided with the sealing element 60 on an inner wall thereof.

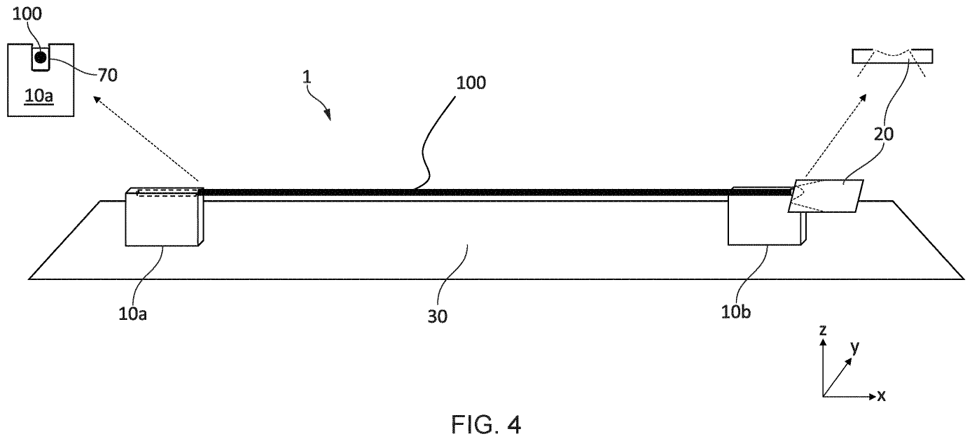

FIG. 4 is a schematic perspective view of a second fluid conduit holder 1 in accordance with some embodiments of the disclosure. Key differences to the first embodiment of FIG. 1 are outlined here. In this second embodiment, the holder 1 comprises just two supports 10: a first support 10a distal from the guide 20 for supporting a distal end of the fluid conduit 100, and a second support 10b for supporting both a proximal end of the fluid conduit 100 in use, and the guide 20. In this embodiment, the first support 10a extends axially beyond the length of the fluid conduit 100 and provides a recess containing a retaining element 70 (see inset cross-section view) to retain the fluid conduit 100 therein. The retaining element 70 may comprise a flexible and/or resilient material that restricts movement of the fluid conduit 100 in the recess, holding it in place. The extension of the first support 10a beyond the end axial length of the fluid conduit 100 assists to protect the distal end of the fluid conduit 100 in use, which may be sharp.

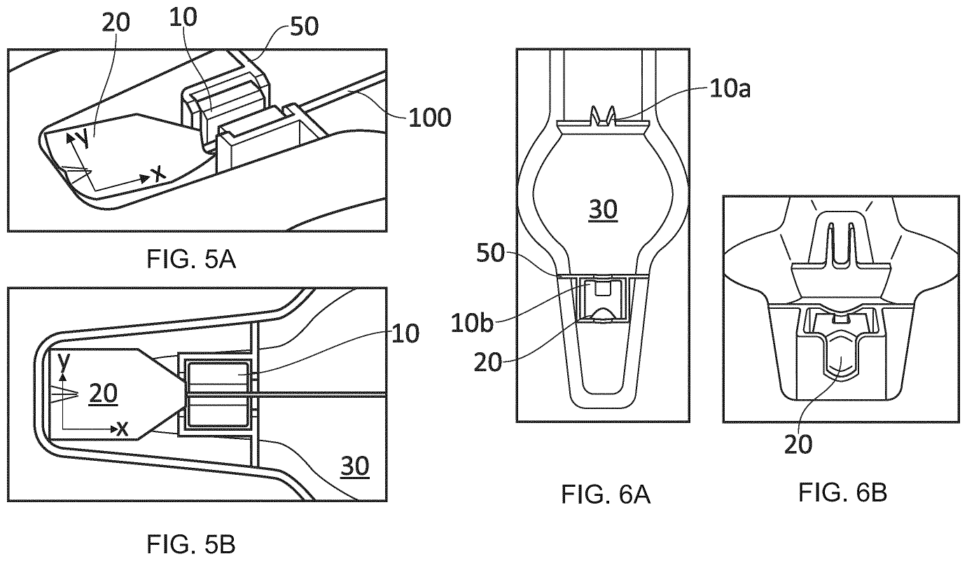

In the embodiment of FIG. 4, the guide 20 comprises a concave surface 20 that tapers (narrows) in width along the y-axis towards the lumen 110. The inset cross-section view shows that the guide 20 is concave in cross-section, perpendicular to an axis of extent of the fluid conduit 100, and effectively functions as a half-funnel. FIGS. 5a and 5b are photographs of the second fluid conduit holder 1, showing the concave surface guide 20 in more detail. These images show that the guide 20 is configured to guide in two perpendicular axes, providing a path to the lumen 110 in use that is guided (restricted) in 3 cartesian degrees of freedom: firstly (best shown in FIG. 4 inset and FIG. 5a) by the tapering of the concave shape which narrows in width (width being along the y-axis) when approaching the lumen 110 (along the x-axis or the z-axis), thus guiding (restricting) movement in both y-directional degrees of freedom; and secondly by the axial extent of the guide 20 along the x-axis—the guide 20 may e.g. be horizontal as in FIG. 4 or slant towards the fluid conduit 100 in the z-axis (shown in FIG. 5a), either way guiding (restricting) movement in the negative z directional degree of freedom (only). The guide 20 of FIGS. 5a-b thus effectively operates like a half-funnel, which does not limit movement in the positive z directional degree of freedom, only limiting movement in the negative z-direction.

FIGS. 6a and 6b are photographs of a third fluid conduit holder in accordance with some embodiments of the disclosure. In this embodiment, the holder 1 comprises a first support 10a which is integrated into the package base 30 and is distal from a second support 10b which comprises the guide 20, both supports 10 having a V-shaped channel recess. As best shown in FIG. 6b, the guide 20 comprises a conical recess in the support 10 at a proximal, axial end thereof, effectively providing an integrated funnel. In use, with the fluid conduit 100 in the channel recesses of the supports 10, the guide 20 thus provides a path to the lumen 110 of the fluid conduit 100 that guides towards the lumen 110 in two perpendicular axes by tapering in two perpendicular axes. Thus, as in FIG. 1, the conical recess tapers in two perpendicular axes and thus provides a path to the lumen 110 that is guided (restricted) in 4 cartesian degrees of freedom.

The holder 1 also comprises an upright 50 in the form of a wall which is integrated into the package base 30, similar to the first support 10a. The upright wall 50 locates and retains the second support 10b and may additionally support the protective tear-off lid 40 of the package for the holder 1 shown in FIG. 2.

In some embodiments, the guide 20 is configured to axially align with the fluid conduit 100 when the conduit 100 is received in the holder 1. In other embodiments, the guide 20 further comprises flexible tubing such as telescopic tubing or bellows (not shown) which allows the guide 20 to be moved out of axial alignment with the fluid conduit 100 (and/or extended), to adjust an entry angle to the guide 20, to further aid the user.

The various embodiments described herein are presented only to assist in understanding and teaching the claimed features. These embodiments are provided as a representative sample of embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilised and modifications may be made without departing from the scope of the claimed invention.

Various embodiments of the invention may suitably comprise, consist of, or consist essentially of, appropriate combinations of the disclosed elements, components, features, parts, steps, means, etc., other than those specifically described herein. In addition, this disclosure may include other inventions not presently claimed, but which may be claimed in future. Protection may also be sought for any features disclosed in any one or more published documents referenced herein in combination with the present disclosure.

### INDEX TO REFERENCE NUMERALS

Any reference numerals in the claims have no impact on the claim scope whatsoever and are simply to assist the reader.
1 Fluid conduit holder
10 Support
20 Guide
30 Package base
35 Package side wall
40 Package film
50 Upright
60 Sealing element
70 Retaining element
100 Fluid conduit
110 Fluid conduit lumen

1.-25. (canceled)

26. A fluid conduit holder comprising a support and a guide, wherein the support is configured to receive and locate a fluid conduit with respect to the guide in use, such that the guide provides a tapering path to a lumen of the fluid conduit.

27. The holder of claim 26, wherein the guide comprises a funnel or cone, or a conical, frustoconical, concave, convex or tapering surface.

28. The holder of claim 26, wherein, in use, the guide provides a tapering path that guides to the lumen in at least 3 cartesian degrees of freedom, and/or the guide provides a tapering path that guides to the lumen in 4 cartesian degrees of freedom.

29. The holder of claim 26, wherein, in use, the guide provides a tapering path that:
a. guides to the lumen in at least two perpendicular axes; and/or
b. tapers towards the lumen in at least two perpendicular axes.

30. The holder of claim 26, wherein the guide comprises a narrow end having an opening dimension of substantially 0.5-50 μm.

31. The holder of claim 26, wherein the guide comprises a narrow end and a wide end, and:
i. the narrow end of the guide is configured to be received within the lumen of the fluid conduit in use; or
ii. the fluid conduit is configured to be received within the narrow end of the guide in use.

32. The holder of claim 26, wherein the guide comprises a sealing element for sealing internally or externally around the fluid conduit in use.

33. The holder of claim 26, wherein the support has a recess therein configured to at least partially receive the fluid conduit in use.

34. The holder of claim 33, wherein a lumen of the guide is substantially concentric with a lumen of the recess.

35. The holder of claim 26, wherein the support comprises a retaining element for retaining the fluid conduit therein in use.

36. The holder of claim 26, wherein the support comprises the guide.

37. The holder of claim 26, wherein the support comprises:
a first support at, near or comprising the guide; and
a second support, distal from the guide.

38. The holder of claim 26, further comprising the fluid conduit.

39. The holder of claim 38, wherein the fluid conduit comprises a needle, a syringe, a pipette or a micropipette.

40. The holder of claim 38, wherein:
c. the fluid conduit has a volume of substantially 0.5-50 μl; and/or
d. the fluid conduit has an inner diameter of substantially 0.5-20 μm.

41. The holder of claim 38, wherein when the holder receives the fluid conduit, and a lumen of the guide is substantially concentric with the lumen of the fluid conduit.

42. A package comprising the fluid conduit holder of claim 26.

43. A set of instructions configured to 3D-print the fluid conduit holder or package of claim 26.

44. A method of filling a fluid conduit using a fluid conduit holder, the holder comprising a support and a guide, the method comprising:
i. locating the fluid conduit with respect to the guide such that the guide provides a tapering path to a lumen of the fluid conduit; and
ii. at least partially filling the fluid conduit with fluid via the guide.

45. A method for fertilising an oocyte in vitro, comprising the method of claim 44, including:
iii. at least partially filling the fluid conduit with sperm and optionally an operating fluid; and
iv. injecting the sperm into the oocyte by ICSI or Piezo-ICSI.
